(19) United States
(12) Patent Application Publication
(10) Pub. No.: US 2025/0180499 A1
(43) Pub. Date: Jun. 5, 2025

(54) SENSOR FOR DETECTING AT LEAST ONE PROPERTY OF A FLUID MEDIUM IN A MEASUREMENT CHAMBER

(71) Applicant: Robert Bosch GmbH, Stuttgart (DE)

(72) Inventors: Gerhard Hueftle, Aspach (DE); Christian Alt, Kornwestheim (DE); Ingo Brauer, Vaihingen (DE); Reinhold Herrmann, Stuttgart (DE); Tobias Lang, Stuttgart (DE)

(21) Appl. No.: 18/703,401

(22) PCT Filed: Oct. 24, 2022
(86) PCT No.: PCT/EP2022/079620

§ 371 Date: Oct. 23, 2024

(30) Foreign Application Priority Data
Nov. 4, 2021 (DE) 10 2021 212 418.6

(51) Int. Cl. G01N 25/18; G01N 33/00; H01M 8/0444

(52) U.S. Cl. CPC G01N 25/18; G01N 33/005; H01M 8/04462

(57) ABSTRACT

A sensor for detecting at least one property of a fluid medium in a measurement chamber, in particular for detecting a H₂ fraction in a measurement gas. The sensor includes at least one sensor element which is designed to detect a thermal conductivity of the fluid medium and for outputting a measurement signal. The sensor further includes a branch element, which defines an interior space, the branch element being designed to branch off a part of the fluid medium from the measurement chamber into the interior space, and at least one diaphragm. The sensor element is fluidically connected to the interior space using the diaphragm.

### BACKGROUND INFORMATION

A large number of sensors, sensor elements and methods for detecting at least one property of a fluid medium in a measurement chamber are described in the related art. In principle, this can involve any properties of a gaseous or liquid fluid medium, wherein one or more properties can be detected. The present invention is described below, without limiting further embodiments and applications, in particular with reference to sensor elements for detecting a gas, in particular a H₂ fraction in a measurement gas.

Sensor elements of the type described here are used in a large number of fields, for example in automotive engineering, process engineering, chemistry and mechanical engineering, in particular for determining gas concentrations. For example, the determination of hydrogen concentrations, for example in an air-hydrogen mixture, plays a major role in the application of hydrogen fuel cell systems. Safety-related applications should also be mentioned here. An air-hydrogen mixture becomes ignitable at a hydrogen fraction of approximately 4%. Sensor elements for detecting hydrogen can be used in hydrogen fuel cell vehicles, for example, in order to detect leaking hydrogen due to damage or defects and to trigger warning signals and/or protective measures by coupling to corresponding systems. Therefore, a plurality of hydrogen sensors are required for each fuel cell vehicle, which sensors are either installed in the exhaust tract (exhaust) or operate under atmospheric conditions (ambient).

A large number of measurement principles can be used for such hydrogen sensors. These include the following measurement principles, among others: heat conduction, catalytic pellistor, electrochemical cell, semiconducting metal oxide, chemiresistor, field-effect transistor.

Sensor elements for measuring thermal conductivity are described, for example, in German Patent Application Nos. DE 10 2005 058 832 A1 and DE 10 2014 202 169 A1.

Despite the advantages of the sensor elements from the related art for detecting at least one property of a fluid medium, they still have potential for improvement. If the thermal conductivity is to be measured with a measuring element consisting of a thin membrane, convection of the exhaust gas past the membrane must be avoided, since this would distort the measurement signal. Instead, the fluid medium at the membrane must be as still as possible. However, in the exhaust tract of a fuel cell, gas flow velocities of up to 100 m/s are present. Additionally, a significant amount of water is produced, which can partially be present as droplets that can also distort the measurements.

### SUMMARY

Within the framework of the present invention, a sensor for detecting at least one property of a fluid medium in a measurement chamber is provided, which at least largely avoids the disadvantages of conventional sensors for detecting at least one property of a fluid medium in a measurement chamber and which offers sufficient sensitivity, measuring range, response time and selectivity with respect to the requirements in automotive engineering.

A sensor according to according to an example embodiment of the present invention for detecting at least one property of a fluid medium in at least one measurement chamber, in particular for detecting a H₂ fraction in a measurement gas, comprises at least one sensor element which is designed for detecting a thermal conductivity of the fluid medium and for outputting a measurement signal.

In principle, the measurement chamber can be any open or closed chamber in which the fluid medium, in particular the measurement gas, is contained and/or through which the fluid medium, in particular the measurement gas, flows.

Within the framework of the present invention, in principle, a sensor element is understood to mean any apparatus that can detect the at least one property of the fluid medium and that can, for example, generate at least one measurement signal corresponding to the detected property, for example an electrical measurement signal such as a voltage or a current. The property can be a physical and/or chemical property, for example. Combinations of properties can also be detectable. In particular, the sensor element can be designed for detecting at least one property of a gas, in particular a H₂ fraction in a measurement gas. Other properties and/or combinations of properties can also be detectable. In particular, the sensor element can be configured for use in a hydrogen fuel cell vehicle.

According to an example embodiment of the present invention, the sensor element is designed, for example, as a sensor chip comprising a heatable measurement membrane. For example, the sensor chip comprises a chip surface. The chip surface has a measurement surface that can be exposed to the fluid medium, and a solid surface. Conductor tracks of a sensor circuit with at least one heating element are applied to the measurement surface. Conductor tracks of the sensor circuit with at least one temperature sensor are applied to the solid surface.

According to an example embodiment of the present invention, the sensor chip can be arranged on a base, in particular a glass base or a silicon base. A connection between the sensor chip and the base can be realized by means of anodic bonding. An access channel can also be formed in the base. For example, the access channel is realized in the form of a bore. The access channel can be produced by scoring or sawing processes or by etching processes. With such a design, it is possible to form a space on the side of the measurement surface facing the base, i.e. the underside of the measurement surface. This space can be designed similar to a cavity, for example.

Within the framework of the present invention, a membrane can be understood as a thin structure that, like a skin or film, has a large surface area in relation to its thickness.

According to an example embodiment of the present invention, the sensor also comprises a branch element, which defines an interior space. The branch element is designed to branch off a part of the fluid medium from the measurement chamber into the interior space.

Within the framework of the present invention, in principle a branch element can be understood as any component that is suitable for discharging a predetermined fraction of the fluid medium from the measurement chamber and directing it into its interior.

The sensor also comprises at least one diaphragm. The sensor element is fluidically connected to the interior space by means of the diaphragm.

Within the framework of the present invention, a diaphragm can be understood as a component that causes a narrowing of the cross-section. For this purpose, the diaphragm has an opening, so that a fluid medium can flow through it. The opening has a significantly smaller cross-sectional area than the regions adjacent to the diaphragm.

In the sensor according to an example embodiment of the present invention, the branch element picks up a part of the fluid medium, which may be in a flow movement, and discharges it in the direction of the sensor element. The fluid medium has convective and diffusive fractions when it passes through the diaphragm. On this flow path, the convective flow is calmed by one or more diaphragms.

The diaphragm can be designed to reduce turbulence of the fluid medium. As a result, the flow of the fluid medium is calmed.

The diaphragm can be designed to reduce convective parts of the fluid medium. As a result, the flow of the fluid medium is calmed and convective fractions in particular are reduced, so that a distortion of the measurement signal is significantly reduced.

The diaphragm can be designed in such a way that the fluid medium reaches the sensor element substantially by means of diffusion. As a result, the movement of the fluid medium at the sensor element is so small that the remaining convective fraction no longer causes a signal error. Accordingly, the fluid medium reaches the sensor element mainly by diffusion. The expression “substantially by means of diffusion” can be understood to mean that the velocity of the fluid medium from the measurement chamber is reduced so much that, in the region around the sensor element, it is only 0.2%, preferably only 0.1% and even more preferably only 0.05% of the velocity in the measurement chamber.

The diaphragm can be designed to substantially prevent heat transfer from the sensor element to the fluid medium. As a result, a distortion of the thermal conductivity measurement is prevented and the remaining convective fraction no longer produces a signal error. The expression “substantially prevent heat transfer from the sensor element to the fluid medium” can be understood to mean that heat transfer is only insignificant. In other words, the flow at the sensor element is so low that heat dissipation from it by a convective part of the fluid medium is so low that the measurement of the thermal conductivity of the fluid medium is only insignificantly influenced; the measurement error in relation to the maximum signal of the sensor element is therefore not greater than 5%, preferably not greater than 3% and even more preferably not greater than 2%. However, it is understood that heat transfer also takes place without convection, namely by heat conduction, since this is the measurement principle of the sensor.

The diaphragm or diaphragms is or are preferably designed in such a way that their opening area reduces the cross-sectional area to 1/10 of the flow cross-section of the adjacent space.

According to an example embodiment of the present invention, the branch element is designed to reduce a flow velocity of the fluid medium when it branches off from the measurement chamber into the interior space. In this way, the branch element picks up a part of the fluid medium, which can be in a flow movement, and discharges it as quickly as possible or over short distances in the direction of the sensor element. As a result, the exchange of the fluid medium and the response time of the sensor element are increased.

The branch element can be designed in such a way that the part of the fluid medium that can be branched off from the measurement chamber into the interior space is no more than 10%, preferably no more than 5% and even more preferably no more than 3% of a volume flow of the fluid medium in the measurement chamber. As a result, only a small but representative fraction of the main flow of the fluid medium flows through the sensor and any water droplets or other solid particles present cannot reach the sensor element due to their inertia.

According to an example embodiment of the present invention, the sensor can also comprise a plurality of diaphragms, wherein the diaphragms are arranged one behind the other as viewed in a flow direction from the measurement chamber to the sensor element in such a way that convective fractions of the fluid medium decrease as viewed in the flow direction. By connecting a plurality of diaphragms in series, the flow of the fluid medium can be calmed to such an extent that the movement of the fluid medium at the sensor element is so small that the remaining convective fraction no longer causes a signal error.

The branch element can be designed substantially as a Venturi nozzle comprising a pick-up tube. Due to the implementation as a Venturi nozzle, large water droplets can fly through the sensor due to their inertia and are thus kept away from the sensor element. Very small droplets may follow the flow, but are then uncritical for the measurement due to their small size. The expression “substantially as a Venturi nozzle” can be understood as a shape similar to a Venturi nozzle.

Within the framework of the present invention, a Venturi nozzle can be understood as a component in the form of a tube piece that is designed with a narrowing of the cross-section, for example by two cones directed towards one another, which are joined at the point of their smallest diameter. A pick-up tube is positioned next to it at this point. The operating principle is based on the fact that when a fluid flows through a Venturi nozzle, the dynamic pressure (stagnation pressure) is at a maximum at the narrowest point of the tube and the hydrostatic pressure is at a minimum. The velocity of the fluid increases in proportion to the cross-sections as it flows into the narrower part, because the same mass flows through the entire tube per time (law of continuity). As a result, the pressure in the pick-up tube, which is located in the narrow part, is reduced. This creates a differential pressure, which can be used in measuring devices or to draw in liquids or gases. For ideal liquids (incompressible and frictionless), the pressure difference is given by Bernoulli's equation. For ideal gases, the extended Bernoulli's equation applies.

According to an example embodiment of the present invention, the branch element can be designed to substantially prevent the entry of liquid droplets and/or particles into the sensor element. The term “substantially prevent” is to be understood as meaning that no droplets and/or particles above a predetermined size reach the sensor element. Only very small droplets or particles, i.e. below the predetermined size, can follow the flow, but are then uncritical for the measurement due to their small size.

The measurement chamber can be a flow tube, in particular an exhaust tube of a fuel cell. Accordingly, the sensor is also suitable for regions with comparatively high flow velocities.

### BRIEF DESCRIPTION OF THE DRAWINGS

Further optional details and features of the present invention are apparent from the following description of preferred exemplary embodiments, which are shown schematically in the figures.

FIG. 1 shows a cross-sectional view of a sensor according to one embodiment of the present invention.

### DETAILED DESCRIPTION OF EXAMPLE EMBODIMENTS

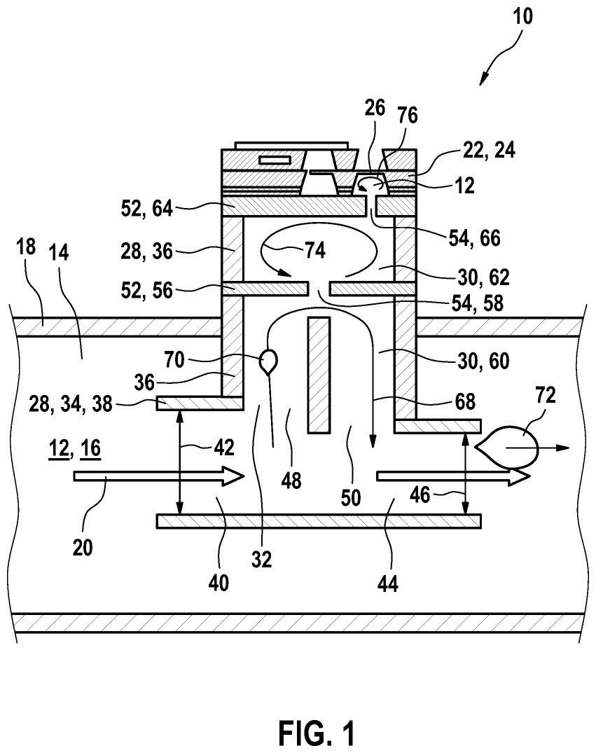

FIG. 1 is a cross-sectional view of a sensor 10 for detecting at least one property of a fluid medium 12 in at least one measurement chamber 14, in particular for detecting a fraction of H₂ in a measurement gas 16. In particular, the sensor 10 can be configured for use in a hydrogen fuel cell vehicle. However, other applications are also possible. In particular, the sensor 10 can comprise one or more further functional elements not shown in the figures, such as, for example, electrodes, electrode leads and contacts, a plurality of layers or other elements. Accordingly, the sensor 10 can be installed in the exhaust tract of the hydrogen fuel cell vehicle (exhaust) or operate under atmospheric conditions (ambient). Consequently, the measurement chamber can be an exhaust tract or interior space of the hydrogen fuel cell vehicle. In the embodiment shown, the measurement chamber 14 is a flow tube in the form of an exhaust tube 18. The sensor 10 is attached, for example screwed, plugged in or welded, to the exhaust tube 18. In the exhaust tube 18, the fluid medium in the form of a gas or a gas mixture containing hydrogen flows in a main flow direction 20. The flow velocity can be up to 100 m/s.

The sensor 10 has a sensor element 22. The sensor element 22 is designed for detecting the thermal conductivity of the fluid medium and for outputting a measurement signal. For this purpose, the sensor element 22 is designed as a sensor chip 24 comprising a heatable membrane 26. The heating of the membrane 26 is carried out by means of a heating element, not shown in detail, which is arranged on the membrane 26. The outputting of the measurement signal is carried out by means of a temperature sensor conductor track, not shown in detail, which is arranged on a solid surface of the sensor chip 24.

The sensor 10 also has a branch element 28. The branch element 28 defines an interior space 30. The branch element 28 is designed to branch off a part of the fluid medium 12 from the measurement chamber 14 into the interior space 30. For this purpose, the branch element 28 has at least one opening 32. Furthermore, the branch element 28 can be connected to the measurement chamber 14. FIG. 1 shows the branch element 28 in the connected state. The branch element 28 is designed to reduce a flow velocity of the fluid medium 12 when branching off from the measurement chamber 14 into the interior space. In other words, the branch element 28 is designed in such a way that a flow velocity of the fluid medium 12 is reduced when it enters the interior space 30. Furthermore, the branch element 28 is designed in such a way that the part of the fluid medium 12 that can be branched off from the measurement chamber 14 into the interior space 30 is not more than 10%, preferably not more than 5% and even more preferably not more than 3% of a volume flow of the fluid medium 12 in the measurement chamber 14. As a result, only a small but representative fraction of the main flow of the fluid medium 12 flows through the sensor 10 and any water droplets or other solid particles present cannot reach the sensor element 22 due to their inertia. In other words, most of the fluid medium from the measurement chamber 14 flows past the sensor 10 and only a part of the part that enters the branch element 28 flows into the interior space 30.

As shown in FIG. 1, the branch element 28 is substantially designed as a Venturi nozzle 34 comprising a pick-up tube 36, for example. The branch element 28 thus has a flow tube 38 that, in the installed state, extends parallel to the main flow direction 20 of the fluid medium in the exhaust tube 18. The flow tube 38 can be flowed through by the fluid medium 12. The flow tube 38 has at least a first tube portion 40 with a first cross-sectional area 42 and a second tube portion 44 with a second cross-sectional area 46. The first tube portion 40 faces the main flow direction 20 and the second tube portion 44 faces away from the main flow direction 20. In other words, the first tube portion 40 is located at the upstream end of the flow tube 38 and the second tube portion 44 is located at the downstream end of the flow tube 38. The first cross-sectional area 42 is larger than the second cross-sectional area 46. It is understood that the flow tube 38 can have further tube portions. For example, the flow tube 38 can have a third tube portion that is located further downstream than the second tube portion 44 and has a third cross-sectional area. The third cross-sectional area is larger than the second cross-sectional area and can, for example, be the same size as the first cross-sectional area.

The pick-up tube 36 defines the interior space 30. In the exemplary embodiment shown, the pick-up tube 36 is oriented substantially perpendicular to the flow tube 38. However, it is explicitly emphasized that other orientations of the pick-up tube 36 are also possible. Therefore, the orientation of the pick-up tube 36 relative to the flow tube 38 is not decisive for the measurement principle of the sensor 10.

The pick-up tube 36 is bent. The pick-up tube 36 is connected to the flow tube 38 in the region of the cross-sectional narrowing of the flow tube 38. The pick-up tube 36 is partially U-shaped. Thus, the pick-up tube 36 has a first opening 48, which is fluidically connected to the first tube portion 40, and a second opening 50, which is fluidically connected to the second tube portion 44.

The sensor 10 also has at least one diaphragm 52. The sensor element 22 is fluidically connected to the interior space 30 by means of the diaphragm 52. Accordingly, the sensor element 22 is not directly connected to the measurement chamber 14, but via the interior space 30 of the branch element 28. The diaphragm 52 is designed to reduce turbulence of the fluid medium 12. The diaphragm 52 is designed to reduce convective parts of the fluid medium 12. In particular, the diaphragm 52 is designed in such a way that the fluid medium 12 reaches the sensor element 22 substantially by means of diffusion. Furthermore, the diaphragm 52 is designed to substantially prevent heat transfer from the sensor element 22 to the fluid medium 12. For this purpose, the diaphragm 52 is designed with a diaphragm opening 54, which has a comparatively small cross-section.

As shown in FIG. 1, the sensor 10 has a plurality of diaphragms 52 in the embodiment shown. The diaphragms 52 are arranged one behind the other as viewed in a flow direction from the measurement chamber 14 to the sensor element 22 in such a way that convective fractions of the fluid medium 12 decrease as viewed in the flow direction. More precisely, in the embodiment shown, the sensor 10 has a first diaphragm 56 having a first diaphragm opening 58, which divides the interior space 30 into a first interior space portion 60 and a second interior space portion 62. The first interior space portion 60 is adjacent to the flow tube 38 or the exhaust tube 18. Furthermore, the sensor 10 has a second diaphragm 64 having a second diaphragm opening 66. The second diaphragm 64 is spaced from the first diaphragm 56 and is adjacent to the second interior space portion 62. The sensor element 22 is fluidically connected to the second interior space portion 62 via the second diaphragm opening 66. The second diaphragm opening 66 has a smaller cross-sectional area than the first diaphragm opening 54.

The operating mode of the sensor 10 is described below. The fluid medium 12 flows in the exhaust tube 18, which represents the measurement chamber 14, at a comparatively high velocity of up to 100 m/s. A part of the fluid medium also flows through the flow tube 38 of the branch element 28. As described above, the pick-up tube 36 is substantially U-shaped in the region of the inner space 30 and, more precisely, of the first inner space portion 60. As a result, the fluid medium 12 is partially branched off from the measurement chamber 14 or the flow tube 38 and thus passes through the first opening 48 into the interior space 30 or initially into the first interior space portion 60, wherein a part of the branched-off fluid medium 12 is deflected by approximately 180° and exits through the second opening 50 back into the flow tube 38, as indicated by an arrow 68. Smaller water droplets 70 or particles can follow this flow deflection, but also exit back into the flow tube 38 or have no influence on the measurement signal of the sensor element 22. Larger water droplets 72 or particles cannot follow the flow deflection and continue to flow with the fluid medium 12 in the main flow direction 20 through the flow tube 38. In this way, the branch element 28 is designed to substantially prevent the entry of droplets and/or particles into the sensor element 22.

Another part of the branched-off fluid medium 12 passes through the first diaphragm opening 58 of the first diaphragm 56 into the second interior space portion 62 by means of diffusion and convection, as indicated by an arrow 74. The fluid medium 12 passes from the second interior space portion 62 to the sensor element through the second diaphragm opening 66 of the second diaphragm 64 predominantly by means of diffusion, as indicated by an arrow 76. Convective flow fractions of the fluid medium 12 in contact with the membrane 26 are not present or only present to a negligible extent, so that the sensor element 22 can carry out an undistorted measurement of the thermal conductivity of the fluid medium 12. Accordingly, a successive reduction in the flow velocity of the fluid medium 12 takes place starting from the measurement chamber 14, via the first interior space portion 60 and the second interior space portion 62 and finally in the region adjacent to the sensor element 22.

1-10. (canceled)

11. A sensor for detecting at least one property of a fluid medium in a measurement chamber, comprising:
at least one sensor element configured to detect a thermal conductivity of the fluid medium and to output a measurement signal;
a branch element that defines an interior space, wherein the branch element is configured to branch off a part of the fluid medium from the measurement chamber into the interior space; and
at least one diaphragm, wherein the sensor element is fluidically connected to the interior space by the diaphragm.

12. The sensor according to claim 11, wherein the sensor is configured to detect a H2 fraction in a measurement gas.

13. The sensor according to claim 11, wherein the diaphragm is configured to reduce turbulence of the fluid medium.

14. The sensor according to claim 11, wherein the diaphragm is configured to reduce convective parts of the fluid medium.

15. The sensor according to claim 11, wherein the diaphragm is configured in such a way that the fluid medium reaches the sensor element substantially by diffusion.

16. The sensor according to claim 11, wherein the diaphragm is configured to substantially prevent heat transfer from the sensor element to the fluid medium.

17. The sensor according to claim 11, wherein the branch element is configured to reduce a flow velocity of the fluid medium when branching off from the measurement chamber into the interior space.

18. The sensor according to claim 11, further comprising a plurality of diaphragms, wherein the diaphragms are arranged one behind the other as viewed in a flow direction from the measurement chamber to the sensor element in such a way that convective fractions of the fluid medium decrease as viewed in the flow direction.

19. The sensor according to claim 11, wherein the branch element is configured substantially as a Venturi nozzle including a pick-up tube.

20. The sensor according to claim 11, wherein the branch element is configured to substantially prevent entry of liquid droplets and/or particles into the sensor element.

21. The sensor according to claim 11, wherein the measurement chamber is an exhaust tube of a fuel cell.
